(19) United States
(12) Patent Application Publication
(10) Pub. No.: US 2025/0134961 A1
(43) Pub. Date: May 1, 2025

(54) ORAL FORMULATION FOR ACTIVATION OF AUTOLOGOUS STEM CELLS

(71) Applicant: Chienyu Investment Co., Ltd., Taipei City (TW)

(72) Inventors: Chai Ching LIN, Yilan County (TW); Cho Chen HSIEH, Taipei City (TW); Ryan LIN, Taipei City (TW)

(73) Assignee: Chienyu Investment Co., Ltd., Taipei City (TW)

(21) Appl. No.: 18/933,789

(22) Filed: Oct. 31, 2024

Related U.S. Application Data
(60) Provisional application No. 63/594,850, filed on Oct. 31, 2023.
(60) Provisional application No. 63/594,858, filed on Oct. 31, 2023.
(60) Provisional application No. 63/594,862, filed on Oct. 31, 2023.

(51) Int. Cl. A61K 38/19; A61K 9/00; A61K 9/113; A61K 35/28; A61K 45/06; A61P 35/00

(52) U.S. Cl. CPC A61K 38/193; A61K 9/0053; A61K 9/113; A61K 35/28; A61K 45/06; A61P 35/00

(57) ABSTRACT

The present invention provides an oral formulation for activation of autologous stem cell, comprising a stem cell activator encapsulated into a water-in-oil-in-water (W/O/W) multiple emulsion; wherein the stem cell activator induces autologous CD34⁺CD45⁺ hematopoietic stem cells (HSCs), wherein the stem cell activator is selected from the group consisting of granulocyte colony-stimulating factor (G-CSF), stem cell factor (SCF) and combination thereof. The W/O/W multiple emulsion protects the stem cell activator from stomach acid digestion, preserving its bioactivity during transport to the bone marrow (BM) via the Peyer's patches of the intestinal lymphatic system.

### CROSS REFERENCE

This Non-provisional application claims the priority under 35 U.S.C. § 119(e) on U.S. Patent Provisional Application Nos. 63/594,850, 63/594,858 and 63/594,862 filed on Oct. 31, 2023, the entire contents of which are hereby incorporated by reference.

### FIELD OF THE INVENTION

The present invention relates to a novel oral formulation for activation of autologous stem cells in a subject.

### BACKGROUND

Antigen-specific cytotoxic T lymphocytes and cellular components of the innate immune system contribute to immune surveillance of malignant cell growth, In particular, natural killer (NK) cells can eliminate abnormal cells without priming or sensitization. Efforts at harnessing the anti-tumor activity of NK cells have been investigated for the immunotherapy of human cancer for over two decades. However, many malignant cells express major histocompatibility complex (MHC) class I antigens and are thus naturally resistant to lysis by autologous NK cells.

NK cell-based immunotherapy has been developed, which provides a practical therapeutic strategy for patients with advanced solid tumors (STs). This approach is adaptively conducted by the autologous and identical NK cells after in vitro expansion and overnight activation. However, the NK cell-based cancer immunotherapy has been faced with some fundamental and technical limitations. Moreover, the desirable outcomes of the NK cell therapy may not be achieved due to the complex tumor microenvironment by inhibition of intra-tumoral polarization and cytotoxicity of implanted NK cells. Currently, stem cells (SCs) technology provides a powerful opportunity to generate more effective and universal sources of the NK cells. Several strategies have been developed to differentiate types of the pluripotent and adult SCs into mature NK cells. these SCs-derived NK cells need the main features including higher cytokine production, intra-tumoral polarization capabilities and stronger anti-tumor properties.

Hematopoietic stem cells (HSC) possess multipotentiality, enabling them to self-renew and also to produce mature blood cells, such as erythrocytes, leukocytes, platelets, and lymphocytes, which are considered to be effective in derivation of NK cells or in enhancing the immunity of a patient. It was found that CD34 and CD45 are markers of human HSC, and all colony-forming activity of human bone marrow (BM) cells.

It is desirable to have a new approach for treating a cancer, a chronic disease or a condition in need of an enhancement of immunity through an enhancement of autologous stem cells (such as HSC) in a patient.

### BRIEF SUMMARY OF THE INVENTION

Accordingly, the present invention provides an oral formulation for activation of autologous stem cells in a subject, which is potential to prepare an composition/pharmaceutical composition as an adjuvant for the treatment of a cancer, a chronic disease, or a condition in need of an enhancement of immunity in a subject, such as aging, by stimulating the growth of stem cells and enhancing the immunity.

In one aspect, the present invention provides an oral formulation for activation of autologous stem cell, comprising:
a water-in-oil-in-water (W/O/W) multiple emulsion; and
a stem cell activator for induction of autologous CD34⁺CD45⁺ hematopoietic stem cells (HSCs), which is selected from the group consisting of granulocyte colony-stimulating factor (G-CSF), stem cell factor (SCF) and combination thereof;
a pharmaceutically acceptable carrier;
   wherein the stem cell activator is encapsulated into the water-in-oil-in-water (WOW) multiple emulsion.

According to the invention, the stem cell activator is encapsulated into the WOW multiple emulsion.

In the invention, the stem cell activator can enter the bone marrow through the intestinal lymphatic system of the subject, thereby enhancing autologous stem cells in the subject, which can improve the treatment of various diseases or conditions, and aging by stimulating the growth of stem cells and enhancing the immunity.

In one example of the invention, the water-in-oil-in-water (W/O/W) multiple emulsion is food-grade emulsion, which comprises an aqueous phase, a lipid phase, and a gel solution, which protects the stem cell activator from stomach acid digestion, preserving its bioactivity during transport to the bone marrow (BM) via the Peyer's patches of the intestinal lymphatic system, ultimately inducing autologous CD34⁺CD45⁺ hematopoietic stem cells (HSCs), and subsequently enhancing the production of NK cells.

In one embodiment of the invention, the water-in-oil-in-water (W/O/W) multiple emulsion is a food-grade emulsion comprising an aqueous phase, a lipid phase, and a gel solution.

In one example of the invention, the oral formulation further comprises mesenchymal stem cells (MSC) exosomes, which significantly boosts the production of autologous HSCs, which enhances the subsequent production of NK cells.

In one example of the invention, the stem cell activator is produced by a culture of Pichia pastoris supplemented with sugar, transform with a plasmid containing the nucleotide sequence of the stem cell activator, such as granulocyte colony-stimulating factor (G-CSF), particularly a recombinant human G-CSF or a modified G-CSF.

According to the invention, the oral formulation is effective as an adjuvant for treatment of a cancer, a chronic disease or a condition in need of an enhancement of immunity in the subject.

In one further aspect, the present invention provides a use of the oral formulation of claim 1 in manufacturing an adjuvant for treatment of a cancer, a chronic disease or a condition in need of an enhancement of immunity in the subject.

In one yet aspect, the present invention provides an adjuvant method for improving a treatment of a cancer, a chronic disease or a condition in need of an enhancement of immunity in a subject, which comprises administering the subject with the oral formulation of claim 1 in combination of the treatment.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The drawings presenting the preferred embodiments of the present invention are aimed at explaining the present invention. It should be understood that the present invention is not limited to the preferred embodiments shown.

FIGS. 1(A), 1(B), 1(C) and 1(D) provide the particle size distribution of the W/O/W multiple emulsions, wherein the FIG. 1(A) shows the light microscope, FIG. 1(B) shows the fluorescence microscope to reflect GFP inside, FIG. 1(C) shows the SEM, FIG. 1(D) shows the schematic diagram, and FIG. 1(E) shows the laser diffraction particle size analyzer, the average diameter of the emulsions was 2.35 μm. (W/O/W: water-in-oil-in-water; GFP: green fluorescent protein; SEM: scanning electron microscopy).

FIG. 2 shows the results of the acid resistance test of GFP in the W/O/W multiple emulsions after incubation in simulated gastric fluid (SGF) at pH 1.2 and simulated intestinal fluid (SIF) at pH 6.8, wherein the symbols represent the remaining fluorescence of GFP within the micro-emulsions compared to GFP without micro-emulsions, and the differences were found to be significant (*** p<0.001).

FIG. 3 shows the results of the stability test of GFP in the W/O/W multiple emulsions at 60° C. for 5 days, which was an accelerated test equivalent to storage at 25° C. for 80 days; wherein the symbols represent the remaining fluorescence of GFP within the emulsions compared to GFP without emulsions (** p<0.01; *** p<0.001).

FIG. 4 shows the penetration rate of GFP within the W/O/W multiple emulsions as compared to GFP only without emulsions, wherein they were co-cultured with Caco-2 cell monolayer, as a model of the intestinal epithelial barrier, at different cultured time. The pictures were taken by fluorescence microscope to show the time point of GFP penetration into the cells.

FIG. 5 shows the comparison of endocytosis of GFP only and GFP encapsulated within W/O/W multiple emulsions in Peyer's patches of mouse small intestinal tissues after 2 hours of oral administration in a live mouse model, wherein the above Peyer's patch photographs were magnified 400 times as shown in (1) light microscope, (2) fluorescence microscope to label microfold cells (M cells) marker Abcam antibody 902, and (3) GFP penetration into the Peyer's patches.

FIGS. 6(A) and 6(B) show the CD34⁺CD45⁺ HSCs in PB detected after oral intake of the designed CD34 Activator emulsions; wherein FIG. 6(A) shows the data of the low-dose group were separately analyzed by gender after 3 months and 6 months; FIG. 6(B) shows the total data of the high-dose group were analyzed (a-c Different letters mean significant differences (p<0.05), as compared on the same row by one-way ANOVA statistical test. N indicates the number of tested individuals).

FIGS. 7(A) and 7(B) show the hypothesized pathway of the orally designed emulsions packaged with the target protein to enhance human autologous HSCs after oral intake based on the GFP emulsions in mice oral study; wherein FIG. 7(A) shows that the GFP emulsions might enter through M cells into the Peyer's patches by endocytosis, then connect to the lymphatic system and lymph nodes; FIG. 7(B) shows the designed emulsions successfully transport and protect the active proteins through the lymphatic pathway between small intestines and bone marrow (BM); if the target protein is the stem cell activator, it should be able to be successfully transported to the bone marrow and induce the proliferation of HSCs in the BM, and release the HSCs into the peripheral blood, which can be measured. In fact, the research findings indirectly support the hypothesis that the designed stem cell activator can successfully induce the concentration of HSCs in human PB after oral administration of the novel emulsion.

FIGS. 8(A) and 8(B) show the improvement obtained by the addition of MSC exosomes to the oral formulation according to the invention, which were tested in term of the expression of 48 kinds of cytokines by two kinds of the RayBio™ Human Cytokine Antibody Array.

FIGS. 9(A) and 9(B) show that the addition of MSC exosomes to the oral formulation according to the invention, provided an enhanced effect in the downstream NK cells of HSCs. FIG. 9(A) shows the percentages in individual NK cells, and FIG. 9(B) shows the statistical significance of the difference by paired t-test (p<0.0001).

FIGS. 10(A), 10(B) and 10(C) provide the expression of rhG-CSF protein by E. coli BL21 Codon Plus® (DE3)-RIPL; wherein FIG. 10(A) shows the agarose gel (1.5%) electrophoresis of PCR-amplified hG-CSF coding DNA sequence (Lane M: DNA markers (Protech, Bio-100 DNA Ladder); lane 1: PCR amplification of hG-CSF (525bp)); FIG. 10(B) shows that hG-CSF was inserted into the plasmid between the BamH1 and Xhol restriction sites; FIG. 10(C) shows the Western blotting analysis of rhG-CSF. Lane M: protein molecular mass markers (Bio-Rad); lane 1: The presence of rhG-CSF was confirmed by anti-hG-CSF monoclonal antibody).

FIGS. 11(A), 11(B) and 11(C) show the protein expression of rhG-CSF by Pichia pastoris; wherein FIG. 11(A) shows the Agarose gel (2%) electrophoresis of PCR amplified hG-CSF coding DNA sequence (Lane M: DNA markers; lane 1:positive control (with hG-CSF DNA); lane 2: pGAPZα B-hG-CSF-E. coli TOP10F′ transformed); FIG. 11(B) shows that hG-CSF was inserted into the pGAPZα B plasmid between the Xho I and Not I restriction sites; FIG. 11(C) shows the Western blotting analysis of rhG-CSF (Lane M: protein molecular mass markers (Bio-Rad); lane 1: The rhG-CSF was confirmed by anti-hG-CSF monoclonal antibody for double-checking).

FIGS. 12(A) and 12(B) provide the comparisons of the bioactivity of rhG-CSF expressed by E. coli and Pichia pastoris respectively, wherein the HL-60 cell proliferation was tested on different concentrations of rh-GCSF using the MTT assay. Each data point was compared to the 0 concentration of each rhG-CSF group, considered as 100% control. It showed that the proliferation around 124-127% level was at the rhG-CSF concentration of 26.75 μg/mL in the E. coli group and 5 μg/mL in the Pichia pastoris group. The bioactivity of rhG-CSF expressed by Pichia pastoris induced by 4% sucrose was almost 5 times that of E. coli.

### DETAILED DESCRIPTION OF THE INVENTION

Unless otherwise defined herein, scientific and technical terms used herein have the meanings that are commonly understood by those of ordinary skill in the art.

As used herein, the singular forms “a”, “an”, and “the” include plural referents unless the context clearly dictates otherwise. Thus, for example, reference to “a sample” includes a plurality of such samples and equivalents thereto known to those skilled in the art.

The present invention provides an oral formulation for activation of autologous stem cell, comprising a stem cell activator encapsulated into a water-in-oil-in-water (W/O/W) multiple emulsion; wherein the stem cell activator induces autologous CD34⁺CD45⁺ hematopoietic stem cells (HSCs), wherein the stem cell activator is selected from the group consisting of granulocyte colony-stimulating factor (G-CSF), stem cell factor (SCF) and combination thereof. The oral formulation is characterized by the W/O/W multiple emulsion, which protects the stem cell activator from stomach acid digestion, preserving its bioactivity during transport to the bone marrow (BM) via the Peyer's patches of the intestinal lymphatic system.

In the invention, stem cell factor (SCF) is used to induce CD34⁺CD45⁺ HSCs and mesenchymal stem cell (MSC) proliferation in the BM and G-CSF is used to mobilize mature stem cells from BM into the bloodstream. Accordingly, the present invention provides a food-grade liquid emulsion to package the combination of G-CSF and SCF, referred as the stem cell activator, for oral consumption. This delivery formulation is designed to protect the stem cell activator.

In embodiments of the inventions, the stem cell activator includes but not limited to peptides, growth factors, cytokines, and any other proteins and/or non-proteins of stem cell activators to stimulate and mobilize autologous CD34⁺CD45⁺ HSCs, thereby enhancing an individual's healing abilities. This formulation is particularly useful for older individuals suffering from chronic diseases such as dementia, Parkinson's disease, stroke, liver failure, kidney failure, osteoporosis, and spinal cord injury and etc., who may require a convenient nutrition supplement to intake the designed emulsion of stem cell activator orally for stem cell regeneration instead of injection.

In one example of the invention, the emulsion may further comprise human mesenchymal stem cell (MSC) exosomes to enhance the downstream production of CD3⁻CD56⁺ natural killer cells (NK) following HSCs induction through oral administration.

In one particular example, the invention provides an oral formulation G-CSF, SCF and MSC exosomes, encapsulated in a W/O/W emulsion that significantly boosts the production of autologous HSCs and subsequently NK cells.

As used herein the term “multiple emulsion”, also known as “double emulsions,” refers to a water-in-oil-in-water (W/O/W) multiple emulsion, which is a three-phase system comprising internal aqueous droplets, oil droplets, and an external aqueous solution. A primary surfactant-stabilizing layer entraps several small internal aqueous droplets within larger oil droplets. Then, a secondary surfactant layer entraps oil droplets with multiple internal droplets, which are dispersed in a continuous external aqueous phase. W/O/W multiple emulsions have properties such as better stability, smaller droplets, higher loading capacity, and controlled release of bioactive substances, making them highly potential for use in pharmaceuticals, cosmetics, and food products. They show great potential for oral delivery of the active ingredient and be used for food-grade oral delivery of the stem cell activator through the intestinal lymphatic system into the BM.

As used herein, the term “subject” or “patient” refers to a human or a non-human mammal, such as primate, rodent, monkey, a pet animal like dog, cat, rabbit and so on.

As used herein, the term “pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for a bioactive activator for stem cell growth is well known in the art.

As used herein, the term “treat,” “treating,” or “treatment” refers to both therapeutic treatment and prophylactic or preventative measures, wherein the object is to prevent, slow down, and/or halt the development of a disease or a impair condition, such as a cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function. For use in therapy, the therapeutically effective amount of the compound is formulated as a pharmaceutical composition for administration.

The present invention is further illustrated by the following examples, which are provided for the purpose of demonstration rather than limitation.

### EXAMPLES

### Example I. Preparation of Water-in-Oil-in-Water (W/O/W) Multiple Emulsion

The W/O/W multiple emulsion was prepared using a two-step emulsification process, with water (A phase) being emulsified in oil (B phase), followed by the emulsification of B phase in a gel solution (C phase). This modified method utilized a volume ratio of 1:1-10:1-100 for the three phases. The food-grade W/O/W emulsion consisted of a stem cell activator in the aqueous phase (A), an oil-in-water interface emulsifier in the oil phase (B), and an acid-resistant colloid (pH 3.0-5.0) in the gel solution (C).

The oil-in-water interface emulsifier claim includes combinations or individual use of Glycerin fatty acid ester, Sucrose fatty acid ester, Lecithin, Span, and Tween. The pH 3.0-5.0 acid-resistant colloid claim includes combinations or individual use of Gar gum, Arabic gum, Xanthan gum, High-ester pectin containing 60-70% esters and Sodium carboxymethyl cellulose. The C phase acts as a gel component, providing thickening, gelling, and stabilizing properties. The A-B-C formulation, regardless of concentration.

### 1. Morphology Observation

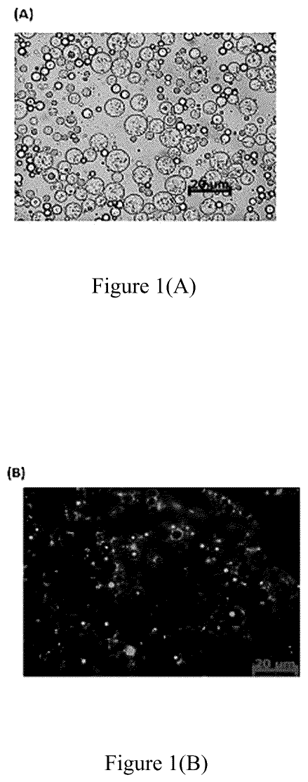

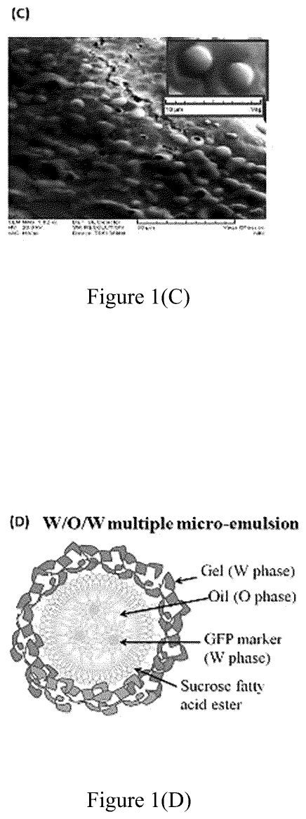

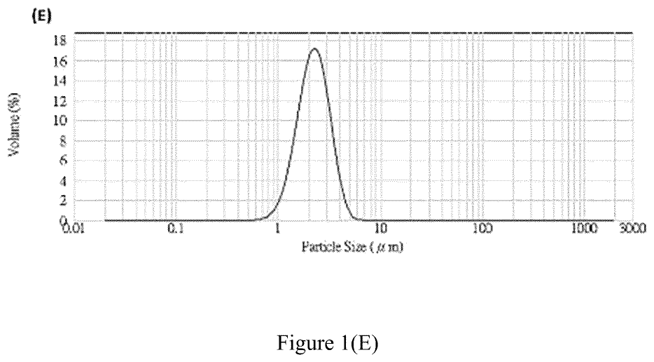

The emulsion samples were observed using an optical microscope (Olympus, BX41; 1,000×) to assess homogeneity and phase separation. FIG. 1 showed that the morphology of the emulsions was highly complex, with numerous droplets located within oil droplets that were dispersed in the gel solution. Additionally, the particle size and morphology of the W/O/W emulsion were confirmed using a scanning electron microscope (SEM; VEGA TS 5136 MM) after a dry process. To prepare the samples for SEM imaging, emulsion samples were diluted 100-fold with water and dropped onto SEM pin stub mounts (Ted Pella Inc., USA) coated with carbon conductive tape (Ted Pella Inc., USA). Dehydration baking was performed in a precision oven for 24 hours at 30° C., and after drying, the samples were sputtered with gold (sputter coater 108) for 2 minutes and scanned at an accelerated voltage of 20 kV. The SEM photographs (FIG. 1) revealed that the emulsion droplets were regular and spherical in shape. To observe the protein distribution in the W/O/W emulsions, inner aqueous phase containing green fluorescent protein (GFP) was prepared and evaluated using fluorescence microscopy (FIG. 1). It was demonstrated that GFP was indeed packaged inside the droplets of the W/O/W multiple emulsions.

### 2. Droplet Size Analysis

The droplet size of the W/O/W emulsion dispersions was determined using a Laser Diffraction Particle Size Analyzer (Malvern). The autocorrelation function of the scattered intensity was obtained from a digital correlator, and the calculated data were analyzed using the program Mastersizer 2000 (Malvern, Worcestershire, UK). The droplet size distribution was unimodal, with a diameter range of 1-10 μm and an average value of 2.35 μm (FIG. 1).

### 3. Acid Resistance

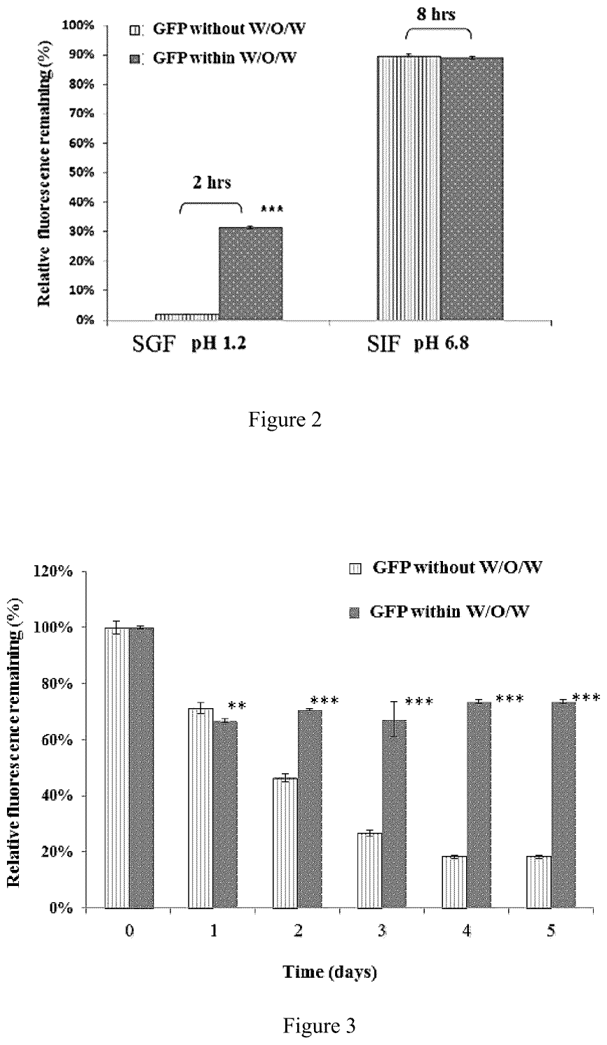

After oral administration, emulsions pass through different parts of the gastrointestinal tract and are exposed to varying pH and enzymatic conditions, which can affect their physicochemical properties and stability. To simulate the pH conditions in the GI tract, GFP packaged inside the emulsions was subjected to simulated gastric fluid (SGF) (without pepsin) and simulated intestinal fluid (SIF) at the pH of the stomach and intestine, respectively. If the GFP fluorescence decreased in SGF or SIF, it indicated GFP degradation. SGF and SIF were prepared according to the 26th United States Pharmacopeia (USP 26). SGF (without pepsin) was prepared by dissolving 2 g of NaCl in 7 mL of 12 N HCl and then adjusting the final volume to 1000 mL with pH adjustment to 1.2. SIF (without pancreatin) was prepared by dissolving 6.8 g of monobasic potassium phosphate in 250 ml of water. Next, 77 mL of 0.2 N NaOH and 500 ml of water were added. The pH was adjusted to 6.8 with 0.2 N NaOH and/or 0.2 N HCl, and the final volume was adjusted to 1000 mL. Two milliliters of 187.5 μg/mL GFP and 187.5 μg/mL GFP emulsion were individually added to 2 mL of either SGF or SIF and placed in screw-capped tubes. The samples were incubated for a specific period in SGF and SIF, which were 2 hours for SGF and 8 hours for SIF. After the period, the fluorescence of each sample was immediately measured by a spectrophotometer (excitation wave: 395 nm, emission wave: 509 nm). The results showed that the fluorescence of GFP emulsion in SGF was able to maintain 31% acid resistance after 2 hours, whereas GFP alone without emulsion was unable to maintain stability (FIG. 2) (p<0.01).

### 4. Accelerated Stability

Accelerated stability was tested under abnormal conditions of high temperature at 60° C. to evaluate long-term stability. For the thermal stress test, GFP only and GFP emulsion were heated at 60° C. for 5 days. Time points were taken at 1, 2, 3, 4, and 5 days. Then, the fluorescence of each sample was immediately measured in triplicate by a spectrophotometer (excitation wave: 395 nm, emission wave: 509 nm). After two days of incubation, it was observed that the percentage of relative fluorescence of GFP emulsions was significantly higher than GFP only (p<0.01) and maintained more than 70% fluorescence for 5 days (FIG. 3). This suggests that the designed W/O/W emulsion can protect the target protein against denaturation at high temperature.

The results were expressed as mean values and standard deviation (Mean±SD). The statistical analysis of data, including pH-dependent stability, accelerated stability, and bioactivity studies, was performed by one-way analysis of variance (ANOVA), followed by Holm-Sidak's post-test for pairwise comparisons. Differences in P-values less than 0.01 were significantly different.

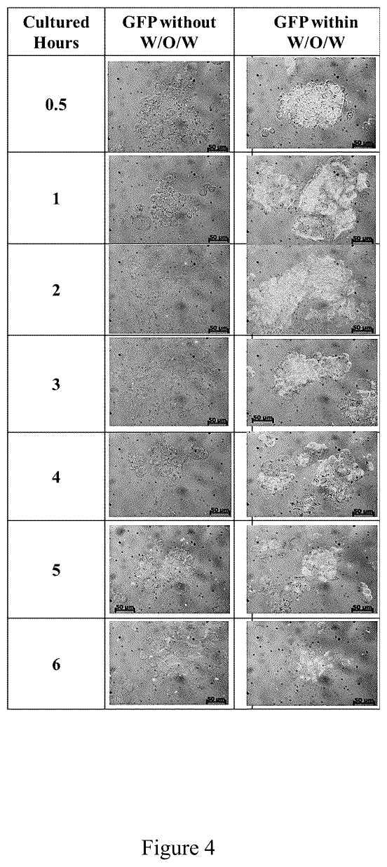

The penetration rate was evaluated using human colon adenocarcinoma, Caco-2 cells (Bioresourse Collection and Research Center, BCRC), as a model for the intestinal epithelial barrier. The cells were cultured in Eagle's Minimum Essential Medium (MEM) (GIBCO, 12000-022; containing 1% non-essential amino acids), supplemented with 20% heat-inactivated fetal bovine serum (Hyclone, SH30070.30). Caco-2 cells were seeded at a density of 6×10⁴ cells/cm² in 6-well culture plates (24 mm diameter) (Nunc, 140675) and allowed to grow in a humidified 37° C. incubator with 5% CO₂. Culture medium was changed every other day until the cells reached 100% confluency. Prior to each experiment, the cell monolayers were rinsed twice with the warm transport buffer (25 mM HEPES, 1 g/L D-glucose and 0.35 g/L sodium bicarbonate in DPBS solution, pH 7.4) and then added in each well for 30 min equilibration at 37° C. After that, different treatments were proceeded, including GFP only (dissolved in buffer) and GFP emulsion (GFP packaged by inner aqueous phase). After periods of incubation, the Caco-2 cells adhered on the plates were washed for three times with warm 1× DPBS (pH 7.4) and were trypsinized away from plates. The pellet was washed twice with 1× DPBS by gently pipetting after centrifugation. Finally, the resuspended cells in 1× DPBS were dropped onto the glass slides for the fluorescent observation (Olympus BX41 microscope). The results showed that GFP packaged in the W/O/W emulsion can easily penetrate through the Caco-2 cell monolayer, like the intestine mucosa, within 0.5 hour (FIG. 4). On the contrary, without emulsions, GFP only didn't have the same efficiency of penetration rate.

### 5. Endocytosis Test in Peyer's Patches of Small Intestine by Oral

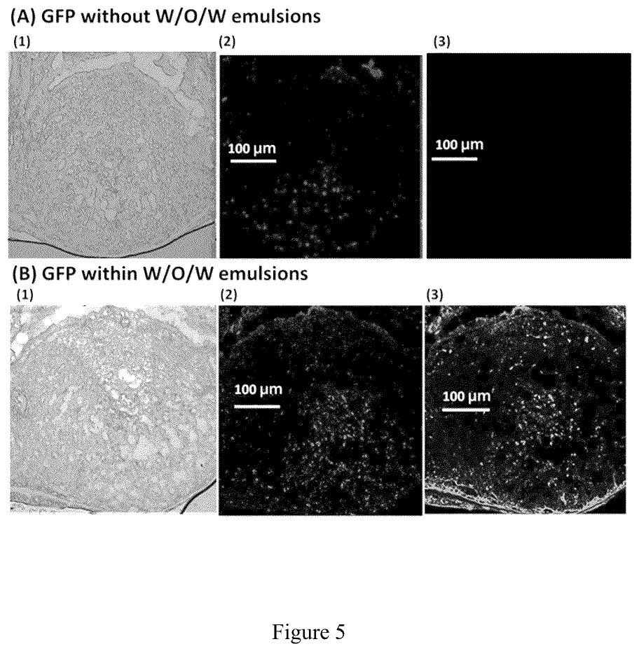

To test endocytosis in Peyer's patches of the small intestine, two groups were created, GFP only without emulsions and GFP within the W/O/W multiple emulsions, after being orally administered to mice in vivo for 2 hours. Photographs of the GFP emulsion group represented the green fluorescence of GFP in the same location as the microfold cells (M cells) marker, Abcam antibody 902, of Peyer's patches, observed at 400× magnification using a light microscope and a fluorescence microscope (FIG. 5). However, the GFP only group did not show any fluorescence. This indicates that the emulsions were successful in protecting the GFP protein and facilitating its absorption via the transport pathway of the Peyer's patches in the small intestinal lymphatic system, rather than the hepatic portal vein circulation.

The emulsions facilitate the target protein's transport from the vein circulation to the intestinal lymphatic system. Notably, Peyer's patches were initially the primary site for lipid internalization in the digestive system, and lipids can be connected to other lymphatic systems such as bone marrow, spleen, and lymph nodes through the intestinal lymphatic system before entering the bloodstream via the thoracic duct. If the target protein present in emulsions can be internalized by Peyer's patches and enter the intestinal lymphatic system, it is likely that the protein will be transported to the bone marrow along with the lymphatic system and maintain its biological activity. The bone marrow is the target tissue that stimulates HSCs and MSCs, and its main signals are our own G-CSF and SCF. If the novel emulsions can successfully protect the growth factors of rhG-CSF and rhSCF and reach the bone marrow through the intestinal lymphatic system via Peyer's patches, it could become a new formulation to promote our own stem cells, HSCs, and MSCs.

This invention confirmed that the GFP target protein in the designed emulsions was internalized into the intestinal lymphatic system via Peyer's patches. However, it is unclear whether the protein will remain active and be transported to the bone marrow. To address this, the next step is to test the growth factor of oral rhG-CSF and rhSCF emulsions in humans and indirectly measure their effect on the peripheral blood HSCs.

### Example II. Evaluating the Oral Formulation in PB

1. Testing the Bioactivities of the Stem Cell Activator Orally can be Demonstrated by Testing the CD34⁺CD45⁺ HSCs in PB.

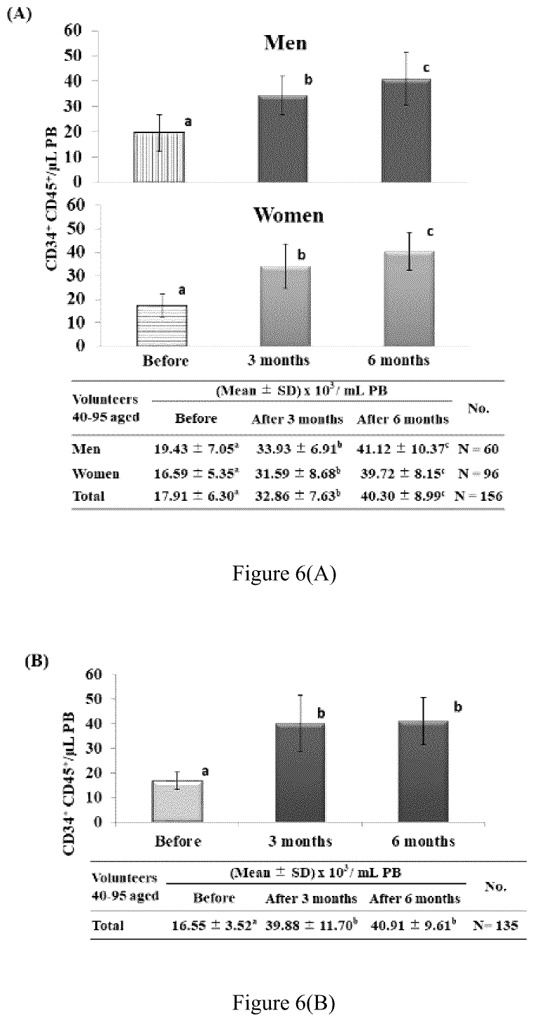

In the oral test, the bioactive rhG-CSF and rhSCF are packaged together into the designed emulsions. For the human trials, we randomly recruited 291 unrelated volunteers (aged 40-95 years) from the community who did not have any specific diseases in clinics. At each age-stage, they were randomly assigned to receive the oral supplement of stem cell activator emulsion once a day, with two groups receiving either low-dose (rhG-CSF+rhSCF: 1-5 μg/kg B.W.) or high-dose (rhG-CSF+rhSCF: 6-30 μg/kg B.W.) treatment. In the low-dose group, the HSC count in peripheral blood significantly increased after 3 months (32.8±7.63×10³/mL PB, p<0.001) compared to before treatment (17.91±6.30×10³/mL PB) and reached the highest level at the 6th month (40.30±8.99×10³/mL PB, p<0.001) (FIG. 6(A)). In the high-dose group of 135 volunteers, their HSC count quickly reached the highest level at the third month (39.88±11.70×10³/mL PB, p<0.001) (FIG. 6(B)).

To analyze the CD34⁺CD45⁺ HSCs, we used a modified method from Preti et al. (20), which combines forward and side light scatter with the intensity of CD34⁺ and CD45⁺ fluorescent antibody staining to accurately enumerate these rare events. This sequential gating method formed the basis of a clinical guideline for CD34⁺ cell enumeration from the International Society for Cellular Therapy (ISCT) (21) and is a featured selection in Current Protocols in Cytometry. The method has since been widely used and validated.

The concentration of HSCs in PB was measured using the BD Accuri C6 Plus flow cytometer (BD Bioscience, San Jose, CA, USA) and analyzed using the BD Accuri C6 Plus software (BD Bioscience, San Jose, CA, USA).

### 2. The Oral Formulation Enters the Bone Marrow Through the Lymphatic Pathway.

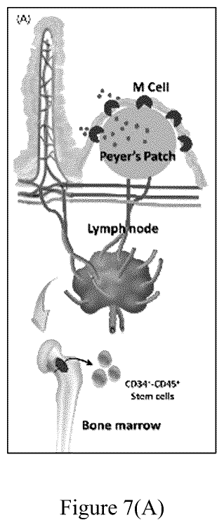

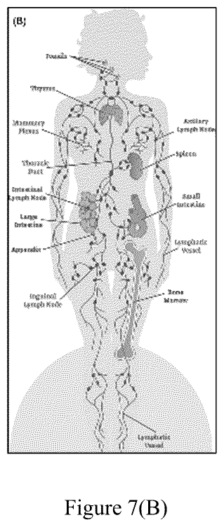

In a mouse study, it was observed that GFP emulsion droplets administered orally could enter the intestinal lymphatic system via M cells in Peyer's patches. Based on this finding, we hypothesize that the designed emulsions may protect target proteins and transport them to the bone marrow through the lymphatic pathway (as shown in FIG. 7). It should be noted that the majority of CD34⁺CD45⁺ HSCs are produced in the bone marrow. If the target protein is the stem cell activator, it should be able to be successfully transported to the BM and induce the proliferation of HSCs in the BM, and release the HSCs into the peripheral blood, which can be measured by collecting blood samples to determine the number of HSCs.

There is a hypothesis that the novel oral formulation of the stem cell activator might enter the bone marrow through the lymphatic pathway. This hypothesis is since the lymphatic system connects with the bone marrow and can transport immune cells and other substances into the bone marrow. If the hypothesis is confirmed, it could provide a new avenue for stem cell activation and potential therapeutic benefits. In fact, our research findings indirectly support the hypothesis that the designed stem cell activator can successfully induce the concentration of CD34⁺CD45⁺ HSCs in human PB after oral administration of the novel emulsion.

### Example III. Test for the Improvement of Chronic Diseases by Oral Intake of the Novel Emulsion of the stem Cell Activator

### 1. Case of Diabetes Wound Repair

A woman who suffered from diabetes for more than ten years developed skin ulcers on her lower limbs that had not healed for many years. After taking oral emulsion with the stem cell activator for six months, the number of stem cells increased, and the wound finally healed. The woman showed a good result after the treatment.

### 2. Case of Renal Function Decline

A woman who suffered from Sjogren's syndrome for more than ten years developed renal function for many years. After taking oral emulsion with the stem cell activator for six months, the number of stem cells increased, and the data of Estimated GFR increase, from 54 to 63. An expectedly result was found in this case.

### 3. Case of Sexual Dysfunction Due to Aging

A man has been 72 years old in 2022. Sexual function began to decline ten years ago. Testosterone level was 323. Ten years later, testosterone level was 416 and 880 after 10 and 18 months of oral administration. His sexual function has recovered to when he was young. This showed an excellent result.

Example III. Addition of MSC Exosomes to Enhance the Function of the Oral Formulation to Increase CD3-CDS6+NK Cells in PB

NK cells differentiate directly from HSCs in certain tissues such as bone marrow, lymph nodes, and spleen and then proliferate on their own. However, additional signals may be required to trigger differentiation and proliferation.

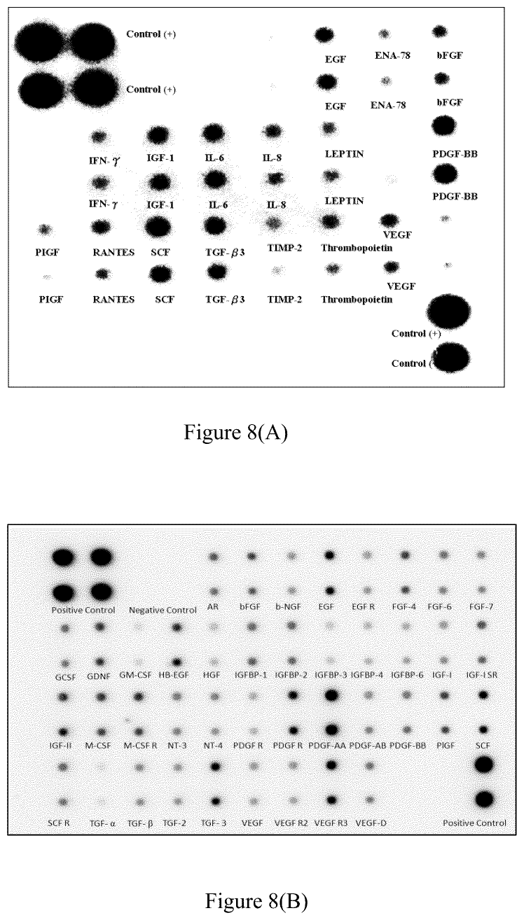

In this example, MSC exosomes were added to evaluate the effects in enhancing the differentiation of HSCs to NK cells, which are downstream cells of HSCs. MSC exosomes contain over 48 cytokines (as shown in FIG. 8) and can act as signals to promote the differentiation and proliferation of NK cells.

The human MSC cell line was obtained from healthy human bone marrow purchased by the National Development and Research Institutes (NDRI) in the USA for research purposes in 2006 and was isolated in our lab. The MSC cells were cultured and subcultured using Dulbecco's Modified Eagle Medium (DMEM) supplemented with 10% fetal bovine serum and 1% antibiotic-antimycotic solution. The cells were preserved in liquid nitrogen for up to 20 passages of subculture.

To obtain MSC exosomes, MSC cells were incubated and passaged for three days. After that, the original medium was replaced with serum-free DMEM supplemented with rhG-CSF+rhSCF (0.01-1 μg/mL) for another three days, during which the MSC cells secreted numerous exosomes into the incubation medium. The medium containing exosomes was collected and tested using the RayBioTM Human Cytokine Antibody Array, which includes over 48 types of cytokines (referred to as MSC exosomes in this invention) (as shown in FIG. 8).

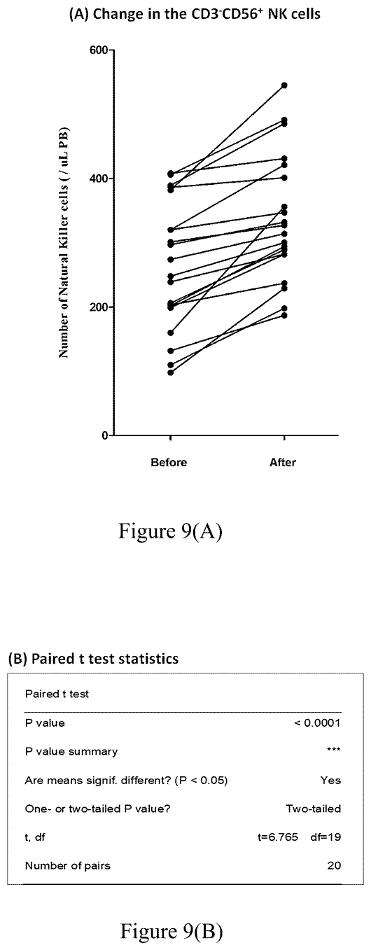

To prepare the novel oral formulation of G-CSF+SCF+MSC exosomes within the designed emulsion, the culture medium containing MSC exosomes was additionally added at a concentration of 0.1%-1.0% (v/v). In a human trial approved by the IRB in the Taipei Hsintien Tzu Chi Hospital (10-M-014), 20 volunteers were randomly selected to use the designed G-CSF and SCF emulsions for three months (referred to as “Before”), followed by G-CSF+SCF+MSC exosomes within the designed emulsion for another three months (After the fourth to sixth month of intake, referred to as “After”) (FIG. 9). The numbers of CD34⁺CD45⁺ HSCs and CD3⁻CD56⁺ NK cells in their PB were measured before and after using the formulations. The data showed a significant increase in the number of CD3⁻CD56⁺ NK cells (paired t-test, p<0.0001), suggesting that MSC exosomes enhanced the differentiation of HSCs into NK cells and the proliferation of NK cells. However, the concentrations of HSCs are not significantly different, compared to “Before” and “After”.

The concentration of HSCs in PB was measured by using the BD Accuri C6 Plus flow cytometer (BD Bioscience, San Jose, CA, USA) and analyzed with the BD Accuri C6 Plus software (BD Bioscience, San Jose, CA, USA). The data were analyzed using linear regression to determine the percent neutralization at a given antibody concentration and the EC50, using GraphPad Prism software (GraphPad Software Inc., San Diego, CA). To compare the plaque reduction neutralization test and flow cytometry neutralization assay, 68 cotton rat serum samples were tested. The linear regression analysis demonstrated a significant correlation, with an R2 value of 0.8444 and a p-value<0.0001.

To compare the individual NK concentration before and after, the statistical analysis was performed using GraphPad Prism software (GraphPad Software, Inc., La Jolla, CA). Paired t-tests were used to compare mean scores “Before” and “After” use. Results were considered statistically significant for p<0.0001.

### Example IV. Preparation of G-CSF using a Pichia pastoris Culture System

A. Production of Higher Bioactivity rhG-CSF

Recombinant human G-CSF is typically produced using the E. coli system in the pharmaceutical industry. However, this invention found the yeast system, Pichia pastoris (purchased from the Bioresource Collection and Research Center, BCRC Number 21531). The yeast system was used to produce rhG-CSF with higher bioactivity. This is achieved by inducing the yeast with a sugar such as sucrose, glucose, fructose, or molasses instead of methanol. This type of food processing using recombinant proteins is generally regarded as safe (GRAS).

### 1. G-CSF Function

The human granulocyte colony-stimulating factor (hG-CSF) plays a significant role in regulating hematopoiesis. G-CSF stimulates the survival, proliferation, differentiation, and function of neutrophil precursors and mature neutrophils.

The production of the active 174 amino acid G-CSF using E. coli was approved by the FDA as a drug in 1991. It is widely used in clinical practice for HSC transplantation and neutropenia treatment. G-CSF is generally co-administered with white blood cell suppression chemotherapy to improve the neutropenic condition. G-CSF has also been used to enhance the number of HSCs after chemotherapy to improve the CD34+stem cells. In clinical therapies for cerebral ischemia, spinal cord injury, bone marrow transplantation, stroke, myocardial infarction, and unilateral ureteral obstruction (UUO), G-CSF is used to induce HSCs mobilization from the bone marrow into the bloodstream to improve disease conditions.

The traditional method of producing rhG-CSF involves using the prokaryotic E. coli system, which may result in issues with protein structure not being the same as that in the human body, including posttranslational modification of glycosylation and the formation of disulfide bonds in Cys36-Cys42 and Cys64-Cys74 to stabilize protein structure and facilitate proper folding. To address these problems, we chose to use the eukaryotic yeast system, Pichia pastoris, as a food-grade protein expression host instead of E. coli. Pichia pastoris has already been established as a protein expression host for producing biopharmaceuticals and industrial enzymes. However, the Pichia pastoris expression system typically produces the target protein by methanol induction, using the alcohol oxidase promoter (AOX promoter). In this example, sugar source induction was used, rather than methanol, to produce a more active rhG-CSF using food-grade yeast, which can be GRAS.

2. Protein Expression of rhG-CSF using E. coli BL21 Codon Plus® (DE3)-RIPL

The U-87 MG cells, a human glioblastoma multiform cell line obtained from the Bioresource Collection and Research Center (BCRC), were cultured in Eagle's Minimum Essential Medium (GIBCO, 11700-077) supplemented with 10% fetal bovine serum (PAA, A15-101). Total RNA was extracted from 1×10⁶ to 1×10⁷ cells using TRI Reagent® RNA Isolation Reagent (Invitrogen, 15596-018) following the manufacturer's instructions. The RNA was quantified using a UV spectrophotometer (UVP BioDoc-ItTM System) and checked for DNA contamination. The RNA was reverse-transcribed into cDNA using the SuperScript III first-strand synthesis system (Invitrogen, 18080-044; 10777-019) with RNAaseOUT (recombinant ribonuclease inhibitor) and Oligo-dT primer. PCR was performed using oligonucleotide primers designed from the hG-CSF nucleotide sequence (Accession number NM_172220). The coding DNA sequence of hG-CSF was amplified using a sense primer (5′-GCCACCCCCCTGGGCCCT-3′) and an antisense primer (5′-GGGCTGGGCAA GGTGGCGTA-3′), followed by 33 cycles of amplification under the following conditions: pre-denaturation at 94° C. for 5 min, denaturation at 94° C. for 1 min, annealing at 57° C. for 50 s, extension at 72° C. for 50 s, and final extension at 72° C. for 5 min. The PCR product was purified by agarose gel extraction and subcloned into the yT&A vector (Yeastern Biotech, Taipei, Taiwan) using YEA T4DNA ligase and single terminal 5′-dT nucleotide overhang for binding the terminal 3′-dA nucleotide overhang. The ligation mixtures were transformed into competent E. coli RR1 cells and grown on LB agar plates containing 50 mg/ml 5-bromo-4-chloro-3-indolyl-b-D-galacto-pyranoside (X-gal) (Promega, V394A), 100 mM isopropylbeta-D-thiogalacto-pyranoside (IPTG) (MDBio, 101-367-93-1), and 50 μg/ml ampicillin (MDBio, 101-69-52-3). The inserts were selected by a color change (blue to white) after 6-8 hrs incubation at 37° C. and identified by PCR analysis on the electrophoresis gel. The coding region for the predicted mature hG-CSF protein was amplified from yT&A-hG-CSF by PCR using a sense primer (5′-CGGGATCCGCCACCCCCCTGGGCCCT-3′, the underline bases indicate the BamHI site) and an antisense primer (5′-CCGCTCGAGGGGCTGGGCAAGGTGGCGTA-3′, the underline bases indicate the Xhol site). The PCR was carried out with 2.5 U of pfu polymerase (Fermentas, EP0571) in a final volume of 50 μl using the conditions. The 525-bp PCR product was purified by agarose-gel extraction and subcloned into the pET-24a (+) expression vector (Novagen) using the BamHI and Xhol restriction enzymes (FIG. 8) (Fermentas, FD0054; FD0694) to yield pET-24a (+)-hG-CSF. The resulting plasmid was transformed into E. coli BL21 Codon Plus® (DE3)-RIPL cells and grown on LB agar plates containing 35 μg/mL kanamycin (MDBio, 101-25389-94-0). A single colony was used to inoculate 5 mL of LB-kanamycin (35 μg/mL) medium in a shaking incubator at 37° C. overnight. The pre-culture was then used to inoculate 500 mL LB-kanamycin (35 μg/mL) medium (inoculum 1% v/v). The culture was grown at 37° C. with vigorous shaking for approximately 3 hours until the exponential phase (0.6<A550 nm<0.8). Protein expression was induced by IPTG at a concentration of 1 mM. After incubation at 37° C. for 2-8 hours, the cells were harvested by centrifugation at 8,000 g for 10 minutes at 4° C. (KUBOTA, 6500). The bacterial culture was pelleted and then resuspended in 50 mL of Buffer A (20 mM Tris-HCl, 10 mM EDTA, 1% Triton X-100, pH 7.5) with 100 μg/mL lysozyme (MDBio, 101-9001-63-2), working at 37° C. for 10 minutes. After that, the cells were processed for several rounds of freezing/thawing and then lysed by sonication (Sonics & Materials, VCX600 model). The cells were sonicated on ice (30×1.5 s pulses with 1 s intervals) and then centrifuged at 13,000× g for 10 minutes at 4° C. The insoluble cytoplasmic fraction may consist of nucleotides, cell debris, and aggregated protein known as inclusion bodies. Hence, inclusion bodies were subjected to repeat centrifugation and wash procedure for three times with 25 mL Buffer A to eliminate endotoxins, proteins, and nucleotides of the host cells. After the washing steps, the pellet was suspended in 25 mL Buffer B (20 mM Tris-HCl, 0.5 M NaCl, 8 M urea, pH 8) and shaken at room temperature for 6-8 hours to dissolve. Residual insoluble materials were removed by centrifuging at 15,000 g for 30 minutes at 25° C. The rhG-CSF was purified by affinity chromatography using a 6x His-tagged tail. The solubilized protein was loaded onto a 10 mL His-Bind column of Ni-charged resin (Novagen, 69670), which was pre-equilibrated in 3 times volume of Buffer B, containing 5 mM imidazole at a flow rate of 2 mL/min. The bound protein was eluted slowly by Buffer B with 800 mM imidazole at the flow rate of 1 mL/min. Finally, the target protein was slowly dialyzed by Buffer D (20 mM Tris-HCl, 0.01 mM EDTA, pH 8).

3. Protein Expression of rhG-CSF in Pichia pastoris by Sugar Induction

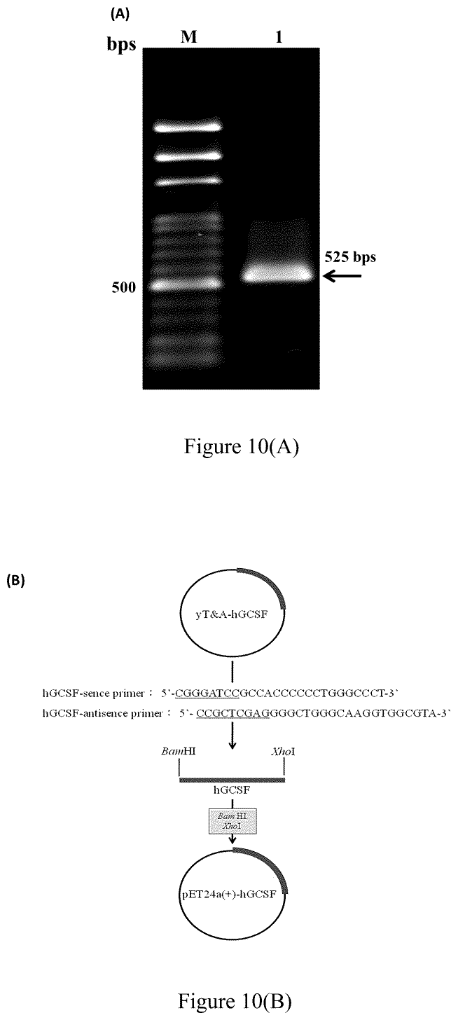

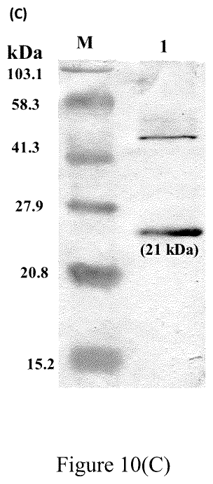

The mRNA of hG-CSF was extracted from human glioblastoma multiform U-87 MG cells (Bioresourse Collection and Research Center, BCRC), and the designed hG-CSF nucleotide sequence (Accession number NM_172220) (see: Sequence Listing) was 525 bps at positions 202 bp to 726 bp. The final 525 bps PCR product was purified from agarose gel and subcloned into the pET24a-hG-CSF plasmid using NotI-HF (New England BioLabs, USA) and FastDigest XhoI restriction enzymes (Fermentas, USA), yielding XhoI-KEX2-STE13-hG-CSF-NotI (561 bps) (FIG. 10). The hG-CSF cDNA was inserted at the restriction sites of NotI-HF and XhoI using the sense primer: 5′-TCGACTCGAGAAAAGAGAGGCTGAAGCTGCCACCCCCCTGGGCCCTGC- 3′, and the antisense primer 5′-GCGGCCGCGGGCTGGGCAAGGTGGCGTA-3′.

The resulting plasmid was ligated using T4 DNA ligase (Fermentas, USA) to form the pGAPZα B (Novagen)-hG-CSF vector (FIG. 10), which was transformed into E. coli TOP1OF′. The constructed E. coli was then transformed into Pichia pastoris GS115 by electroporation using 1.5 kV for 5 ms to create the yeast strain pGAPZα B-hG-CSF-Pichia pastoris GS115, with the glyceraldehyde-3-phosphate dehydrogenase (GAPDH) promoter (pGAP).

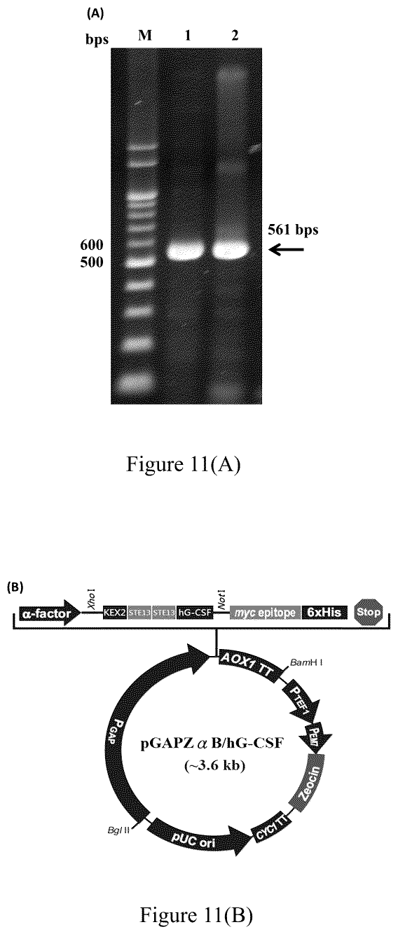

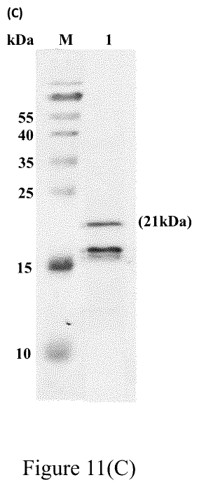

To identify the rhG-CSF protein produced by the yeast strain, it was cultured in YPD medium and induced by 1% yeast extract, 2% meat peptone, and 4% sucrose at 37° C. with shaking at 250 rpm for 12 hrs. The supernatant was collected after centrifugation at 13,000 g and 4° C. for 10 min and then treated with 0.8× volumes of 100% ethanol at −80° C. for 2 hours, followed by drying at 55° C. for 10 min. Protein expression was identified by 15% SDS-PAGE analysis using 2× volumes of SDS loading dye and Western blotting with primary antibody (anti-G-CSF antibody, Cat. No. GF05, Abcam, USA) and secondary antibody (goat anti-Mouse IgG AP conjugate, Cat. No. SAB-101, Stressgen, Canada) (FIG. 11). Western blotting data was analyzed using the CCD ImageQuantTM LAS 4000 (GE Healthcare life Sciences) and ImageQuant TL Software (FIG. 11).

4. To Compare the Bioactivity of rhG-CSF Expressed by E. coli and Pichia pastoris

The bioactivity of rhG-CSF was tested using an in vitro study of human promyelocytic leukemia (HL-60) cells obtained from the Bioresource Collection and Research Center (BCRC). The test method was modified from Yamaguchi et al.⁽²⁷⁾. HL-60 cells were cultured for 2-3 generations in 20% fetal bovine serum (FBS) (PAA, A15-101) of Iscove's Modified Dulbecco's Medium (IMDM) (GIBCO, 12200-036) and then replaced by IMDM containing 1.25% dimethyl sulfoxide (DMSO) without FBS. Different concentrations (0, 1.56, 3.13, 6.25, 12.5, 25, 50, 100 μL/mL) of rhG-CSF produced by E. coli BL21 Codon Plus® (DE3)-RIPL or by Pichia pastoris GS115were added to the 24 wells of 1×10⁵ cells/mL/well to co-culture for 3 days at 37° C. with 5% CO₂. Cell proliferation was measured using 3-(4,5-dimethylthiazol-2-yl)-2,5-diphenyl tetrazolium bromide (MTT) at 0.5 mg/mL for 4 hours at 37° C. with 5% CO₂. After 12 hours of incubation with 500 μL of 10% SDS, 5% isopropanol and 0.012 M HCL/well at 37° C. with 5% CO₂, the supernatant was tested by OD570, using a microplate reader (SpectuaMax Molecular Devices Corp M2).

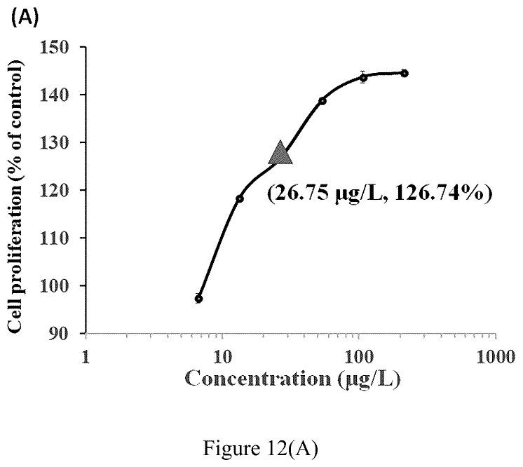

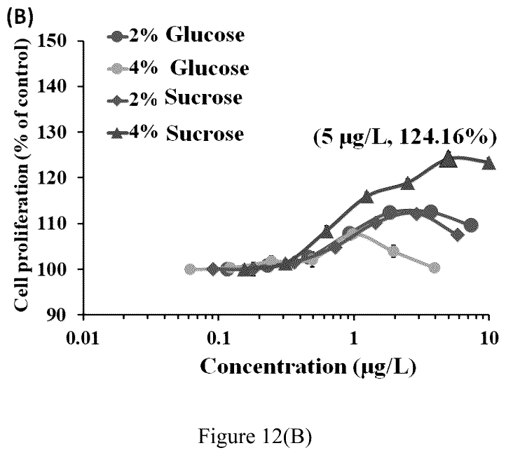

The data for each group was compared to the 0 concentration of rhG-CSF, which was considered as 100% of the control. The results showed that the proliferation of cells at a concentration of 26.75 μg/mL in the E. coli group and 5 μg/mL in the Pichia pastoris group was around 124-127% level. It was found that the bioactivity of rhG-CSF expressed by Pichia pastoris was approximately five-fold higher than that expressed by E. coli (FIG. 12).

### Example V. Clinical Study of the Oral Formulation According to the Invention

### Case 1: Stage IV Cancer Patient

The patient was a male born in 1973. He was diagnosed as a stage IV cancer in January 2022, and the patient underwent tracheotomy and chemotherapy surgery in February 2022. They started eating in March and didn't experience many discomfort symptoms during chemotherapy, such as nausea, hair loss, and insomnia. The patient was discharged in November 2022 with 31.06/μL PB stem cells and 355/μL PB NK cells, and he had already recovered to the level of a healthy person.

Case 2: Stage III Lung Adenocarcinoma with Metastasis to Lymph Nodes

Ms. Zhang, a 54-year-old patient with stage III lung adenocarcinoma with metastasis to lymph nodes, started chemotherapy at the end of August. The fifth cycle of chemotherapy was scheduled for October 25th, but it was temporarily suspended due to a low white blood cell count (less than 1990). On October 27th, she started taking an oral medication. Five days later, on November 1st, her white blood cell count increased to 5650, allowing her to resume chemotherapy to fight the cancer cells.

### Case 3: Stage III THYROID CANCer

Ms. Huang, 73 years old, was diagnosed with stage III thyroid cancer in June 2022. After the diagnosis, she started taking the oral formulation since then. She did not undergo chemotherapy but received targeted drug therapy. However, the targeted drug therapy only lasted for a month and not continued. During the period of taking the oral formulation, Ms. Huang's cancer index was examined four times at the China Medical University, and it decreased from 2313.52 ng/ml on 2022 Oct. 13 to 848.10 ng/ml on 2023 Dec. 7.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

What is claimed is:

1. An oral formulation for activation of stem cells in a subject, comprising:
a water-in-oil-in-water (W/O/W) multiple emulsion;
a stem cell activator for induction of autologous CD34⁺CD45⁺ hematopoietic stem cells (HSCs), which is selected from the group consisting of granulocyte colony-stimulating factor (G-CSF), stem cell factor (SCF) growth factors and combination thereof; and
a pharmaceutically acceptable carrier;
wherein the stem cell activator is encapsulated into the water-in-oil-in-water multiple emulsion.

2. The oral formulation of claim 1, wherein the water-in-oil-in-water (W/O/W) multiple emulsion is food-grade emulsion comprises an aqueous phase, a lipid phase, and a gel solution, which protects the stem cell activator from stomach acid digestion, preserving its bioactivity during transport to the bone marrow (BM) via the Peyer's patches of the intestinal lymphatic system in the subject.

3. The oral formulation of claim 1, further comprising mesenchymal stem cells (MSC) exosomes, which significantly boosts the production of autologous HSCs, which enhances the subsequent production of NK cells.

4. The oral formulation of claim 1, wherein the stem cell activator is produced by a culture of Pichia pastoris supplemented with sugar, transform with a plasmid containing the nucleotide sequence of the stem cell activator.

5. The oral formulation of claim 4, wherein the stem cell activator is granulocyte colony-stimulating factor (G-CSF).

6. The oral formulation of claim 5, wherein the G-CSF is recombinant human G-CSF.

7. The oral formulation of claim 5, wherein the G-CSF is a modified G-CSF.

8. The oral formulation of claim 1, which is effective as an adjuvant for treatment of a cancer, a chronic disease or a condition in need of an enhancement of immunity in the subject.

9. An adjuvant method for improving a treatment of a cancer, a chronic disease or a condition in need of an enhancement of immunity in a subject, which comprises administering the subject with the oral formulation of claim 1 in combination of the treatment.
